(19) United States
(12) Patent Application Publication
(10) Pub. No.: US 2015/0306063 A1
(43) Pub. Date: Oct. 29, 2015

(54) COMPOSITION AND METHOD FOR VAGINAL THERAPY

(71) Applicants: Diane McGINNIS, Williamsport, PA (US); Karen STOPPER, Williamsport, PA (US); Deborah DAY, Williamsport, PA (US); Candice TATE, Williamsport, PA (US)

(72) Inventors: Diane McGINNIS, Williamsport, PA (US); Karen STOPPER, Williamsport, PA (US); Deborah DAY, Williamsport, PA (US); Candice TATE, Williamsport, PA (US)

(21) Appl. No.: 14/695,861

(22) Filed: Apr. 24, 2015

Related U.S. Application Data
(60) Provisional application No. 61/984,337, filed on Apr. 25, 2014.

(51) Int. Cl. A61K 31/325; A61K 9/00

(52) U.S. Cl. CPC A61K 31/325; A61K 9/0034

(57) ABSTRACT

Provided are a composition and method for vaginal therapy. The composition includes a delivery compound and a pharmaceutically active agent dispersed in the delivery compound. The pharmaceutically active agent includes a centrally acting muscle relaxant. The method for vaginal therapy includes preparing a composition including a pharmaceutically active agent dispersed in a delivery compound, and administering the composition intravaginally. The pharmaceutically active agent includes a muscle relaxant. Another method includes administering the composition extravaginally and then administering the composition intravaginally.

### CROSS-REFERENCE TO RELATED APPLICATIONS

This application claims the benefit of and priority to U.S. Provisional Patent Application No. 61/984,337 filed on Apr. 25, 2014, which is hereby incorporated by reference in its entirety.

### FIELD OF THE INVENTION

The present invention is directed to a composition and a method for vaginal therapy. More specifically, the present invention is directed to a composition and a method for vaginal dryness, painful intercourse (dyspareunia), and vaginismus therapy.

### BACKGROUND OF THE INVENTION

At some point in their lives, many women may experience vaginal dryness, dyspareunia, and/or vaginismus (primary or secondary). Vaginismus is an involuntary spasm of the pelvic muscles, particularly the pubococcygeus muscle, which results in an involuntary tightening of the vagina. The bulbocavernosus muscle, and others, may also be involved. Due to the involuntary tightening, it is often difficult and painful, if not impossible, to engage in any type of vaginal penetration, such as sexual intercourse.

Frequently, dyspareunia, vaginismus and vaginal dryness result from a loss of the female hormones estrogen and progesterone, which usually occurs from menopause or hysterectomy. A common treatment for dyspareunia, vaginismus, and vaginal dryness related to menopause includes oral or vaginal hormone replacement therapy. However, hormone replacement therapy may have serious side effects, such as blood clots, and is often not available to those with a history of breast cancer.

Additionally, doctors may refer women with vaginismus and painful intercourse issues for mental health counseling, which often makes women feel as if what seems to be a physical problem is actually a mental health issue. Another treatment includes advising patients to purchase products designed to stretch the opening of the vagina. However, this is often ineffective as the vagina continues to go into spasm upon entry of an object.

A composition and method that show one or more improvements in comparison to the prior art would be desirable in the art.

### BRIEF DESCRIPTION OF THE INVENTION

In an embodiment, a composition for vaginal therapy includes a delivery compound and a pharmaceutically active agent dispersed in the delivery compound. The pharmaceutically active agent includes a centrally acting muscle relaxant.

In another embodiment, a method for vaginal therapy includes preparing a composition including a pharmaceutically active agent dispersed in a delivery compound, and administering the composition intravaginally. The pharmaceutically active agent includes a muscle relaxant.

In another embodiment, a method for vaginal therapy includes preparing a composition including a pharmaceutically active agent and a delivery compound, the preparing comprising dispersing the pharmaceutically active agent in the delivery compound, administering the composition extravaginally, and then administering the composition intravaginally. The pharmaceutically active agent includes a centrally acting muscle relaxant.

An advantage of exemplary embodiments is that the pharmaceutically active agent is delivered transmucosally without moisture from an individual. More specifically, the pharmaceutically active agent is delivered transmucosally from the viscous substance administered intravaginally to an individual experiencing vaginal dryness.

Another advantage is that the composition provides moisture to the vagina and reduces or eliminates the pain of intercourse due to involuntary vaginal tightening.

Yet another advantage is that the composition reduces or eliminates involuntary vaginal tightening.

Still another advantage is that the composition reduces or eliminates the pain of intercourse due to involuntary vaginal tightening and vaginal dryness without hormone therapy and without affecting a sexual partner.

Other features and advantages of the present invention will be apparent from the following more detailed description, taken in conjunction with the accompanying drawings which illustrate, by way of example, the principles of the invention.

### DETAILED DESCRIPTION OF THE INVENTION

Provided are an exemplary composition and method for moisturizing a vagina and/or reducing or eliminating the pain of vaginal penetration due to involuntary vaginal tightening, which may be caused by spasms of the muscles around the vagina. Although described primarily with respect to compositions administered to individuals experiencing vaginal dryness, the invention is not so limited and the compositions may also be administered to individuals without vaginal dryness. Such individuals include, without limitation, women experiencing vaginismus for reasons other than menopause.

In one embodiment, the composition includes a pharmaceutically active agent dispersed in a delivery compound. The pharmaceutically active agent includes any element, substance, or compound for reducing or eliminating the pain of intercourse due to involuntary vaginal tightening. For example, in one embodiment, the pharmaceutically active agent includes a muscle relaxant, such as, but not limited to, a centrally acting muscle relaxant. Other pharmaceutically active agents include, but are not limited to, directly acting muscle relaxants.

The centrally acting muscle relaxant includes any drug or other agent that acts in the central nervous system (CNS). Although the exact mechanism of action of centrally acting muscle relaxants is not known, it may be due to CNS depression, which includes, for example, blocking receptors in the brain. In one embodiment, the centrally acting muscle relaxant includes, for example, an antispasmodic muscle relaxant, such as, but not limited to, methocarbamol. The term “antispasmodic” is considered to be synonymous with the term “spasmolytic”, and as such, the terms are used interchangeably throughout this application. Other examples of centrally acting muscle relaxants include, but are not limited to, carbamic acid esters (e.g., phenprobamate, carisoprodol, styramate, febarbamate); oxazol, thiazine, triazine, and/or derivatives thereof (e.g., chlormezanone, chlorzoxazone); ethers (e.g., orphenadrine); other centrally acting agents (e.g., cyclobenzaprine, metaxalone, tolperisone, mephenesin, mephenoxalone, eprisone, fenyramidol, promoxolane); and/or antispasticity agents (e.g., baclofen, tizanidine).

Different centrally acting muscle relaxants may be selected based upon various characteristics of an individual. For example, in one embodiment, methocarbamol is selected for elderly patients, including patients older than 65 years of age. Methocarbamol is a centrally acting carbamic acid ester having the chemical formula 3-(2-methoxyphenoxy)-1, 2-propanediol 1-carbamate, which includes a half-life that is only slightly prolonged in the elderly as compared with a younger population. Other pharmaceutically active agents may be selected based upon other characteristics, such as health and/or pre-existing conditions.

To reduce or eliminate the pain of intercourse due to involuntary vaginal tightening from administration of the composition, a therapeutically effective amount of the pharmaceutically active agent is dispersed in the delivery compound. As used herein, the “therapeutically effective amount” of the pharmaceutically active agent is an amount which is sufficient to reduce or eliminate the pain of intercourse due to involuntary vaginal tightening. A dosage for providing the therapeutically effective amount may be adjusted depending upon age, physical condition, weight, extent of involuntary vaginal tightening, frequency of treatment, desired extent of effect, and/or any other relevant parameter. In one embodiment, for example, the therapeutically effective amount of the pharmaceutically active agent, such as methocarbamol, includes between 1 and 2000 mg, between 1 and 1500 mg, between 1 and 1000 mg, between 500 and 1500 mg, between 1 and 500 mg, between 500 and 1000 mg, between 1 and 250 mg, between 1 and 100 mg, between 1 and 50 mg, between 1 and 25 mg, between 1 and 10 mg, up to about 6 mg, up to about 3 mg, or any combination, sub-combination, range, or sub-range thereof.

In one embodiment, the delivery compound includes any vehicle for delivering the active agent, such as a solvent and/or a viscous substance. Suitable delivery compounds include, but are not limited to, liquids, lubricants, creams, foams, ointments, oils, gels, pastes, liniments, or lotions. The pharmaceutically active agent is dissolved or dispersed in the delivery compound as a powder, a liquid, in microcapsules, or any other form for being delivered transdermally and/or transmucosally from the delivery compound. Dissolving or dispersing the pharmaceutically active agent in the delivery compound facilitates delivery of the pharmaceutically active agent. For example, in one embodiment, the pharmaceutically active agent is dissolved in the delivery compound, which is administered topically (e.g., intravaginally, to the skin surrounding the vagina) to an individual. The pharmaceutically active agent is then delivered from the delivery compound transdermally and/or transmucosally, depending on where the delivery compound is administered. In another example, microcapsules containing the pharmaceutically active agent are carried by the delivery compound, which is administered topically to an individual. The microcapsules release the pharmaceutically active agent, which is then delivered transdermally and/or transmucosally. After delivering the pharmaceutically active agent from the delivery compound, the pharmaceutically active agent acts to reduce or eliminate symptoms associated with vaginismus, such as involuntary muscle spasms and painful intercourse. For example, reducing or eliminating the pain associated with vaginal penetration may help a woman to relax, which may reduce the involuntary muscle spasms.

When administered intravaginally to individuals lacking the moisture required to dissolve a tablet or capsule, such as individuals experiencing vaginal dryness, the delivery compound facilitates transmucosal delivery of the pharmaceutically active agent. In another embodiment, when administered to an individual experiencing vaginal dryness, the delivery compound lubricates the vagina. In one embodiment, the delivery compound includes a moisturizing and/or emollient component. For example, one delivery compound includes coconut oil. Other examples of delivery compounds having a moisturizing and/or emollient component include oil-in-water emulsions; water-in-oil emulsions; water-soluble lubricants; emulsions including cetyl alcohol, silicone-derivatives, grape seed oil, or dimethicone; argan oil; jojoba oil; palm oil; olive oil; or combinations thereof.

Intravaginal administration of the delivery compound including the moisturizing and/or emollient component moisturizes the vagina while simultaneously facilitating transmucosal delivery of the pharmaceutically active agent. Additionally or alternatively to including moisturizers, the composition may include an element for stimulating fluid production. Elements for stimulating fluid production include, but are not limited to, estrogen, progesterone, or ospemifene.

In one embodiment, the composition includes a compound that enhances absorption or penetration of the pharmaceutically active agent. Absorption enhancing compounds include compounds that enhance skin permeation, such as, by altering the stratum corneum lipids and/or proteins, or by increasing partitioning of the pharmaceutically active agent into the stratum corneum. Examples of absorption enhancing compounds include, but are not limited to, oleic acids or non-ionic surfactants. In another embodiment, the composition includes an analgesic for relieving pain associated with vaginal dryness and/or vaginismus. Suitable analgesics include any transdermal or transmucosal analgesic, such as, but not limited to, buprenorphine, salicylate, capsaicin, or lidocaine.

Administration of the composition is performed prior to penetration of the vagina. The administration prior to penetration provides sufficient time for the pharmaceutically active agent to reduce or eliminate the pain of intercourse due to involuntary vaginal tightening. A timing of the administration is dependent upon at least the pharmaceutically active agent selected, the route of administration, and the dosage of the pharmaceutically active agent. For example, in one embodiment, the composition including methocarbamol is administered 20 to 30 minutes prior to vaginal penetration. In another embodiment, administration of the composition prior to sexual intercourse reduces or eliminates the pain of intercourse due to involuntary vaginal tightening without affecting a sexual partner. In a further embodiment, administration of the composition prior to sexual intercourse reduces or eliminates the pain of intercourse due to involuntary vaginal tightening without interfering with the woman or her sexual partner achieving orgasm.

In one embodiment, as previously discussed, the composition is administered intravaginally. In another embodiment, the delivery compound is administered extravaginally (i.e., topically outside the vagina), such as, to the skin surrounding the vagina. To reduce or eliminate the pain of involuntary vaginal tightening, the delivery compound is topically administered separate from, prior to, and/or simultaneously with intravaginal administration. For example, when intravaginal administration is difficult and/or painful due to involuntary vaginal tightening, the delivery compound is first administered extravaginally. The pharmaceutically active agent is delivered transdermally from the extravaginally administered delivery compound to reduce or eliminate the pain of involuntary vaginal tightening. After the extravaginal administration, the delivery compound may then be administered intravaginally without the pain and difficulty of the involuntary vaginal tightening.

The composition alternatively includes other components for releasing the pharmaceutically active agent therefrom, such as pearls, gel capsules, tablets, pills, or patches. In one embodiment, the composition includes both the delivery compound and one or more of the other components. For example, the composition may include the pharmaceutically active agent dispersed in the viscous substance, which is contained within a gel capsule. In one embodiment, administration of the other components includes intravaginal insertion of the gel capsule, for example, and subsequent release of the pharmaceutically active agent contained therein. When inserted intravaginally into an individual suffering from vaginal dryness, the pharmaceutically active agent is released by any method other than dissolution from vaginal fluids. Such methods include, but are not limited to, rupturing the gel capsule, extruding the pharmaceutically active agent through apertures (e.g., micropores), or dissolving the gel capsule from within. For example, in another embodiment, the gel capsule is inserted within the vagina where the natural body temperature degrades the gel capsule to release the pharmaceutically active agent. In another example, the other component is inserted simultaneously with a moisturizer or lubricant to provide moisture for dissolving the other component (e.g., gel capsule, tablet, pill).

Based upon the delivery compound and/or other components used, the composition is administered from any suitable container. Suitable containers include, but are not limited, jars, tubes, bottles, dispensers, and/or packets. For example, the composition including the viscous substance may be administered from tubes or individual packets.

The following examples are included for clarification only, and are not intended to limit the scope of the disclosure.

### Example 1

In one example, the composition included 500 mg of methocarbamol dispersed in 414 ml of organic coconut oil. After mixing the methocarbamol and the organic coconut oil, half teaspoon and one teaspoon doses of the composition were applied intravaginally 20 to 30 minutes prior to sexual intercourse. Each half teaspoon of the composition included 3 mg of methocarbamol, and each teaspoon of the composition included 6 mg of methocarbamol. On average, one or two doses of the composition were applied, although some women required an increased number of doses. Due to the large mucosal surface of the vagina, the composition was easily absorbed to reduce or eliminate the pain of intercourse due to involuntary pelvic muscle tightening. If more lubrication was needed after administration of the composition, coconut oil without methocarbamol was applied intravaginally.

### Example 2

In one example, the composition includes 500 mg of methocarbamol mixed in 414 ml of organic coconut oil. Half teaspoon doses of the composition are applied to the skin surrounding the vagina starting about 30 minutes prior to vaginal penetration to reduce the pain of vaginal penetration due to involuntary vaginal tightening. Then, starting about 20 minutes prior to vaginal penetration, half teaspoon doses of the composition are applied intravaginally to further reduce or eliminate the pain of vaginal penetration due to involuntary vaginal tightening. If more lubrication is needed, any personal lubricant without methocarbamol is applied as desired.

### Example 3

In one example, the composition includes 144 mg of methocarbamol dispersed in 4 ounces (118 ml) of a water-based, water-soluble personal lubricant, such as, but not limited to K-Y Jelly, which is available from Johnson & Johnson in New Brunswick, NJ. Half teaspoon and one teaspoon doses of the composition are applied intravaginally, each half teaspoon including 3 mg of the methocarbamol, and each teaspoon of the composition including 6 mg of the methocarbamol. The composition is applied 15 to 20 minutes prior to sexual intercourse to provide lubrication and reduce or eliminate the pain of intercourse due to involuntary vaginal tightening.

### Example 4

In one example, the composition includes 6 mg of methocarbamol dispersed in individual containers of personal lubricant. The individual containers include varying amounts of personal lubricant to provide a desired amount of lubricant having a consistent amount methocarbamol.

While the invention has been described with reference to one or more embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A composition for vaginal therapy, the composition comprising:
a delivery compound; and
a pharmaceutically active agent dispersed in the delivery compound;
wherein the pharmaceutically active agent includes a centrally acting muscle relaxant.

2. The composition of claim 1, wherein the centrally acting muscle relaxant comprises an agent that acts in the central nervous system.

3. The composition of claim 2, wherein the centrally acting muscle relaxant is selected from the group consisting of a carbamic acid ester, oxazol, an oxazol derivative, thiazine, a thiazine derivative, triazine, a triazine derivative, an ether, an antispasticity agent, cyclobenzaprine, metaxalone, toperisone, mephenesin, mephenoxalone, eprisone, fenyramidol, promoxolane, and a combination thereof.

4. The composition of claim 3, wherein the carbamic acid ester is selected from the group consisting of methocarbamol, phenprobamate, carisoprodol, styramate, and febarbamate.

5. The composition of claim 4, wherein the carbamic acid ester is methocarbamol.

6. The composition of claim 3, wherein the oxazol derivative, the thiazine derivative, and the triazine derivative are selected from the group consisting of chlormezanone and chlorzoxazone.

7. The composotion of claim 3, wherein the ether is orphenadrine.

8. The composition of claim 3, wherein the antispasticity agent is selected from the group consisting of baclofen and tizanidine.

9. The composition of claim 1, further comprising an absorption enhancing compound selected from the group consisting of oleic acids and non-ionic surfactants.

10. The composition of claim 1, further comprising at least one additive selected from the group consisting of a moisturizer, an emollient, and an analgesic.

11. The composition of claim 1, wherein the delivery compound is selected from the group consisting of a solvent, a viscous substance, a liquid, a lubricant, a cream, a foam, an ointment, an oil, a gel, a paste, a liniment, and a lotion.

12. The composition of claim 1, further comprising a component selected from the group consisting of a pearl, a gel capsule, a tablet, a pill, and a patch.

13. A method for vaginal therapy, comprising:
preparing a composition including a pharmaceutically active agent dispersed in a delivery compound; and
administering the composition intravaginally;
wherein the pharmaceutically active agent includes a muscle relaxant.

14. The method of claim 13, further comprising administering the composition extravaginally prior to the administering of the composition intravaginally.

15. The method of claim 13, wherein the administering of the composition reduces involuntary vaginal tightening.

16. The method of claim 13, wherein the preparing of the composition includes dispersing a therapeutically effective amount of the pharmaceutically active agent in the delivery compound.

17. The method of claim 16, wherein the pharmaceutically active agent comprises methocarbamol and the therapeutically effective amount comprises between 1 and 2000 mg.

18. The method of claim 13, wherein the muscle relaxant comprises a centrally acting muscle relaxant.

19. The method of claim 13, wherein the delivery compound is selected from the group consisting of a solvent, a viscous substance, a liquid, a lubricant, a cream, a foam, an ointment, an oil, a gel, a paste, a liniment, and a lotion.

20. A method for vaginal therapy, comprising:
preparing a composition including a pharmaceutically active agent and a delivery compound, the preparing comprising dispersing the pharmaceutically active agent in the delivery compound;
administering the composition extravaginally; and then
administering the composition intravaginally;
wherein the pharmaceutically active agent includes a centrally acting muscle relaxant.
